(19) United States
(12) Patent Application Publication
(10) Pub. No.: US 2024/0280481 A1
(43) Pub. Date: Aug. 22, 2024

(54) SYSTEM AND METHOD FOR ASSESSING CONCENTRATION OF MOLECULES CONTAINING PROTEIN IN ALGAE BASED ON SPECTRAL MEASUREMENTS

(71) Applicant: CARMEL HAIFA UNIVERSITY ECONOMIC CORPORATION LTD., Haifa (IL)

(72) Inventors: Anna BROOK, Haifa (IL); Niva TADMOR SHALEV, Givataim (IL); Alvaro ISRAEL, Hertzliya (IL)

(21) Appl. No.: 18/566,753

(22) PCT Filed: Jun. 2, 2022
(86) PCT No.: PCT/IL2022/050589

§ 371 Date: Dec. 4, 2023

Related U.S. Application Data
(60) Provisional application No. 63/196,295, filed on Jun. 3, 2021.

(51) Int. Cl. G01N 21/3563; G01N 21/35; G01N 33/00; G06N 20/00

(52) U.S. Cl. CPC G01N 21/3563; G01N 33/0098; G06N 20/00; G01N 2021/3595

(57) ABSTRACT

A method and a system for assessing the concentration of molecules containing protein in algae is disclosed. The method comprising: receiving spectral measurements of a sample containing the algae at a range of wavelengths associated with one or more pigments; preprocessing the spectral measurements; and determining the concentration of the molecules containing protein in the algae, based on the preprocessed spectral measurements, wherein the molecules containing protein consist of at least one or more of; protein and protein bounded to one or more nonprotein molecules.

### CROSS-REFERENCE TO RELATED APPLICATIONS

This application claims the benefit of priority of U.S. Provisional Patent Application No. 63/196,295, titled “HIGH THROUGHPUT AUTOMATED PHENOTYPING TECHNOLOGY FOR SEAWEED BIOCHEMICAL COMPOSITION BY MEANS OF FIELD SPECTROSCOPY AND MACHINE LEARNING ALGORITHM”, filed Jun. 3, 2021. The contents of the above applications are all incorporated by reference as if fully set forth herein in their entirety.

### FIELD OF THE INVENTION

The present invention relates generally to a system and method for assessing the concentration of molecules containing protein in algae. More specifically, the present invention relates to a system and method for assessing the concentration of molecules containing protein in algae-based on spectral measurements.

### BACKGROUND

Industrialized agriculture and over-exploitation of marine resources contribute to the threats of global climate change, population growth, and natural resource degradation which, in turn, affect food security, including the supply of high-quality protein for food. The transition from animal-based proteins to alternative sources, particularly plant-based, could alleviate health issues by significantly reducing potable water use, greenhouse gas emissions, and land clearing. Therefore, there is a growing interest in developing novel protein sources, including edible seaweed.

Seaweeds are known for their high quality and yields of potentially edible protein in proportion to their dry weight. In addition, seaweeds are regarded as an important source for nutrients, vitamins, minerals, and trace elements with broad commercial applications (e.g., food, feed, phycocolloids, fertilizers, pharmaceuticals, nutraceuticals, cosmetics) and environmental benefits. In spite of this, the proportion of seaweed proteins within the total human protein intake is negligible, especially in western countries. Yet, the European Commission has highlighted in a recent report (EC 2020) the significant role of algae (both microalgae and seaweeds) in the development of the bioeconomy sector and the sustainability of food systems. In general, proteins are responsible for many of the functional properties of food and are a major factor in food nutrition assessment. Protein production from seaweeds strongly depends on the sustainability and efficiency of the production processes in preserving nutrition and health benefits, maximizing yield while reducing cost, eliminating waste, and minimizing environmental footprint. Such aspects are derivatives of species selection, cultivation approach, and seaweed biochemical state estimation. Monitoring and detecting desirable traits of seaweed before harvesting are indissolubly related to precision culture and remote sensing technology, both of which are absent from the current seaweed production practices.

There are more than 30,000 recognized seaweed species and they have been classified into three Phyla based on their dominant pigmentation: Red (Rhodophyta), Brown (Ochrophyta) and Green (Chlorophyta). In nature, seaweeds can be found usually within the intertidal zone as well as at the open sea. Seaweed production has exhibited a rapid growth over the last decade. In 2018, marine and costal seaweed production contributed ca. 51% (32 million ton) of the global aquaculture sector, mainly for food and for the hydrocolloids industry. Among species, red and brown seaweeds contributed 53.5% and 46% of the total seaweed production, respectively. Many studies have highlighted the biochemical composition of seaweed that can be exploited for nutritional purposes. Previous studies also explored the substantial variability in desirable traits between and within groups of seaweeds and even within individual species. Fluctuation in biochemical composition can occur temporally and spatially in accordance to light intensity, temperature, nutrients availability and other biotic and abiotic conditions. For instance, protein content variation of the edible green macroalgae Ulva spp., a widespread species, ranged according to literature from 3.7% up to 32.7% on a dry weight basis. Variation in protein content of Gracilaria spp. spp. (Rhodophyta) also been described in the literature from 6.9% of Gracilaria spp. changgi dry-weight (DW), up to 45% of Gracilaria spp. gracilis dry matter. Variability can be explained through algal efficient adaptation and acclimation to a specific environment. One of the greatest challenges for commercialization is controlling or moderating such fluctuations, thus preserving the quality and homogeneity of the functional properties in the yield. Successfully controlling seaweed cultivation within an exposed marine environment is difficult, yet vital for preserving the nutritional properties and maximize productivity. Some studies have suggested to control fluctuations through adequate site selection for extensive seaweed farm. In other studies, pre-deployment of treatments during hatchery period to the structure of the supporting substrates, (e.g. ropes, rigs or rafts) at cultivation stage within the open marine environment, were recognized as crucial in achieving productivity control. Attempts to control seasonality impacts on seaweed productivity and chemical composition have been based mostly on selecting the right season for cultivation. it was suggested to Integrated Multi-Trophic Aquaculture (IMTA) combining fish cages and seaweed as a model to increase biomass productivity, and protein and carbohydrate concentration in offshore operation. The economic feasibility of offshore farms is however uncertain, as evidenced by the absence of operational active seaweed farms within the Western countries.

Previous studies on seaweed land-based operations and nutrient supply optimization have addressed productivity and bio-composition quality mostly in the green seaweed Ulva spp. Studies investigated the effect of solar radiation and spectrum environments on photosynthesis and daily growth rate. The impact of seasonality on growth, yield and nutritional composition (e.g. protein, lipid, ash, and amino acid content) were addressed as well. Additional investigations tested optimal cultivation conditions through system configuration. Vertical stacking of multiple layers was used to increase productivity of Ulva tepida in a land-based system. Flat-panel photobioreactors with controlled temperature were tested to achieve year-round cultivation of Ulva, and drip irrigation platforms associated with a range of fertilizers concentrations were tested for growth response, protein content and areal biomass productivity. Seaweed exergy efficiency was tested of light conversion into biomass using a photobioreactor as a method to increase biomass yield. In addition, protein concentration variability was recorded as a function of cultivation configuration, nitrogen supplement regime, seasonality, region and harvest time. Seaweed biomass with improved protein concentration and better nutrition functionality, necessitates the development of controlled and innovative cultivation protocols and management tools to increase production efficiency and preserve protein quantity and quality.

The ability to increase protein yield at minimum cost is challenging with regard to seaweed biomass. Proteins are present in diverse forms and locations in seaweed; therefore, fractionation is also challenging. Carbohydrate-attached proteins and pigment-attached proteins that are found in seaweed as well as second metabolites hinder protein availability and require additional steps in extraction. This challenge is one aspect that fostered the biorefinery concept in which different non-protein seaweed fractions can be processed and utilized as well for side-stream valorization. Seaweed proteins consist of significant amounts of essential amino acid (EAA) and the total amino acid composition is very similar to ovalbumin. In general, protein content is low in brown seaweed (3-29% dw), moderate in green seaweed (9-32% dw) and can constitute up to 47% of the red seaweed dry weight, which is similar to soybean. However, the low availability exhibits much lower yield after protein purification (10-11% yield from seaweed in comparison to 50-60% yield from soybean). It has been suggested to focus on enriched and functional fraction process as more realistic approach. Either way, higher protein concentration in raw seaweed all year around will increase protein or protein fraction yield at downstream processing. Spectral measurements can provide precise indicative high-throughput non-distractive tools for detecting the seaweed state on-site and assess the protein concentration.

Plant phenotyping is a comprehensive assessment of plant traits complex (e.g., growth rate, physiology, ecology, yield) and the basic measurements of individual quantitative parameters. It has been widely used in agriculture, for instance for leaves, fruits and roots characteristics, and lately also in macroalgae for species selection and carbohydrate detection. Imagery technique aims to measure the phenotype quantitatively through the interaction between light and biomass reflectance, transmittance and absorbance wavelength properties and estimate crop state. Methods to collect imagery data can be done by remote sensing such as commercial satellite and aerial air craft tools that give different spectra aspects. However, there is a tradeoff between high temporal and low spatial resolution.

Field spectroscopy approach measures point-by-point spectral radiance using portable spectrometer. Main advantages are its low cost, high resolution temporally and spatially and wide wavelength range across the visible IR (VIS), near-IR (NIR) and shortwave IR (SWIR). Remote sensing includes also multispectral and hyperspectral tools that usually measures nutrient status, growth rate assessment, yield and biomass map. Attenuated Total Reflection (ATR) Fourier Transform Infrared (FTIR) spectroscopy imagery can be used as a tool to learn about functional group molecules, including protein identification and quantification, and protein structural composition.

Currently very little is known about the relationship and dynamics occurring between the visible pigmentation of the seaweed thallus and the prevalence of protein in the seaweed biomass.

Accordingly, there is a need for a direct simple assessment of protein concentration and protein fraction concentration in algae. Such an assessment can be conducted using spectral measurements of visible thallus pigmentation of algae.

### SUMMARY OF THE INVENTION

Some aspects of the invention are related to a method of assessing the concentration of molecules containing protein in algae comprising: receiving spectral measurements of a sample containing the algae at a range of wavelengths associated with one or more pigments; preprocessing the spectral measurements; and determining the concentration of the molecules containing protein in the algae, based on the preprocessed spectral measurements, wherein the molecules containing protein consist of at least one or more of; protein and protein bounded to one or more nonprotein molecules.

In some embodiments, the range of wavelength associated with one or more pigments is determined by identifying in spectral measurements a range of wavelength associated with one or more pigments. In some embodiments, determining the concentration of the molecule containing protein is based on chemical measurements of the amount of molecule containing protein associated with spectral measurements, stored in a database. In some embodiments, the chemical measurements are nitrogen concentration measurements in the algae.

In some embodiments, determining the concentration of the molecule containing protein comprises applying a pretrained, first machine-learning (ML) model on the preprocessed spectral measurements to predict the concentration of the molecules containing protein in the algae. In some embodiments, the first ML model is pre-trained based on an annotated training dataset, comprising a plurality of preprocessed spectral measurements and a corresponding plurality of annotations, representing concentrations of the molecules containing protein in the algae.

In some embodiments, the method further comprises, identifying in the preprocessed spectral measurements at least one type of molecules containing protein. In some embodiments, the identification is based on Attenuated Total Reflection (ATR) Fourier Transform Infrared (FTIR) spectroscopy analysis associated with preprocessed spectral measurements, stored in a database. In some embodiments, identifying comprises: performing FTIR spectroscopy analysis on spectral measurements to produce identification of types of molecules containing protein; and applying a pretrained, second ML model on the preprocessed spectral measurements, to predict at least one of the type of molecules containing protein in the alga.

In some embodiments, wherein the pigment is phycobiliprotein, chlorophyll/carotenoid-binding complexes (LHCs) a and b, and xanthophylls. In some embodiments, the phycobiliproteins is selected from: pink/purple-colored phycoerythrin (PE), blue colored phycocyanin (PC), and bluish-green colored allophycocyanin (APC).

In some embodiments, receiving the spectral measurements is at a wavelength range of 400 to 2500 nm. In some embodiments, receiving the spectral measurements is at a wavelength range of 500-800 nm.

In some embodiments, the method further comprises calculating the pigment concentration based on calculating a ratio between a reflectance coefficient and the scattering coefficient from the spectral measurements.

In some embodiments, the method further comprises: determining growth parameters for growing the algae based on the determined protein concentration and protein-fraction concentration.

Some additional aspects of the invention are directed to a system for assessing the concentration of total cellular protein in algae, comprising: a spectrometer; and a controller configured to execute methods according to embodiments of the invention disclosed herein.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a schematic illustration of a land-based seaweed cultivation system according to some embodiments of the present invention;

FIG. 1B is a block diagram of a system for assessing the concentration total cellular protein in algae according to some embodiments of the invention;

FIG. 1C is a flowchart of a method of assessing the concentration total cellular protein in algae according to some embodiments of the invention;

FIG. 2 is an image of a non-limiting example of a layout of an experimental system according to some embodiments;

FIG. 3 is a diagram showing seawater temperature and irradiance in the cultivation tanks in an experimental environment, from Jun. 23, 2020, to Jul. 20, 2020, according to some embodiments of the invention;

FIG. 4 shows indices imagery of Gracilaria spp. thalli submerged and on top the cover net and on-site reflectance measurements. The highest protein content was demonstrated by seaweed sample from pool 7 (5.56% DW), and the lowest from pool 1 (1.5% DW) in the experiment environment, and SWIR/NIR reflectance measurements of samples pigment solutions from Pools 1 and 7 of the experiment environment, according to some embodiments of the invention;

FIG. 5 shows the daily growth rate (%) of Gracilaria spp. per treatment, in the experiment environment: A1, A2 represents the control group with no addition of external nutrients and with one or two layers of net cover (respectively). B1, B2, and C1, C2 are treatments with addition and intensified addition of nutrients (respectively) covered with one or two layers of net, according to some embodiments of the invention;

FIGS. 6A, 6B, 6C, and 6D show preprocessing process of spectral measurements (a)-(c) and a diagrammatic representation of a prediction model (d), according to some embodiments of the invention;

FIG. 7 shows graphs comparing directly measured protein vs. predicted protein of at various wavelengths according to some embodiments of the invention;

FIG. 8 shows images of substantial variability in desirable traits between and within groups of seaweeds, according to some embodiments of the invention;

FIG. 9 shows a comparison of algae growth rate and protein content at various pools according to embodiments of the invention,

FIG. 10 shows a graphical summary of model prediction performances, according to some embodiments of the invention; and

FIGS. 11A to 11E illustrate spectral measurements preprocessing method to be used in an ML model for assessing protein content in seaweed and a graphical representation of the ML model, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Some aspects of the invention may be directed to a system and a method of assessing at least one of: protein concentration and protein fraction concentration in algae based on spectral measurements. The spectral measurements may be taken directly from algae samples on the field, using, for example, a portable spectrometer. The samples may be taken directly from the cultivation container, several times during the growth of the algae. In some embodiments, the spectral measurements may provide information regarding the types and the concentration of the protein and/or the protein fraction.

In some embodiments, a method according to embodiments of the invention may include data collecting and a training stage in which direct chemical composition measurements taken from the algae samples may be associated with spectral measurements taken from the same algae samples. The direct chemical composition measurements may allow for to calculate the protein and/or protein fraction concentration in the algae. Additionally, Fourier-transform infrared spectroscopy (FTIR) may be conducted on the algae samples in order to identify the types of protein and/or protein fraction included in the samples. The FTIR analysis may also be associated with spectral measurements. In some embodiments, the direct chemical composition measurements and the FTIR analysis can be used as labels in training a machine learning (ML) model to determine the concentration and/or the type of protein and/or protein fraction in algae from spectral measurements.

Attenuated Total Reflection (ATR) Fourier Transform Infrared (FTIR) spectroscopy imagery was used to identify protein molecules absorbing bands in various types of algae.

As used herein “Algae” refers to any type of algae or microalgae, for example, types that can be grown in cultivation systems, either on-land of off-shore. For example, the algae may include, Maacrocystis pyrifera, Lessonia spicata, Gracilaria spp. persica, or any type of Spirulina, Chlorophyceae (Green algae), Phaeophyceae (Brown Algae) Rhodophyceae (Red Algae) micro and/or macro algae, and/or cyanobacteria.

As used here “molecules containing protein” include protein and protein bounded to one or more nonprotein molecules. Some examples for proteins may include peptides, enzymes (e.g., alkaline phosphatase; Rubisco, etc.), glycoproteins and lectins (e.g., carbohydrate-binding proteins), cell wall-attached proteins (e.g., arabinogalactan proteins (AGPs); hydroxyproline-rich glycoproteins (HRGP), etc.), Phycobiliproteins (PBPs), mycosporine-like amino acids (e.g., form secondary metabolites), and the like.

As used herein “protein bounded to one or more nonprotein molecules” may include clusters (anatomic structures where the proteins reside) within the alga cells which include proteins bounded (by any type of chemical bound) to other nonprotein molecules, such as, lipids, polysaccharides, etc.

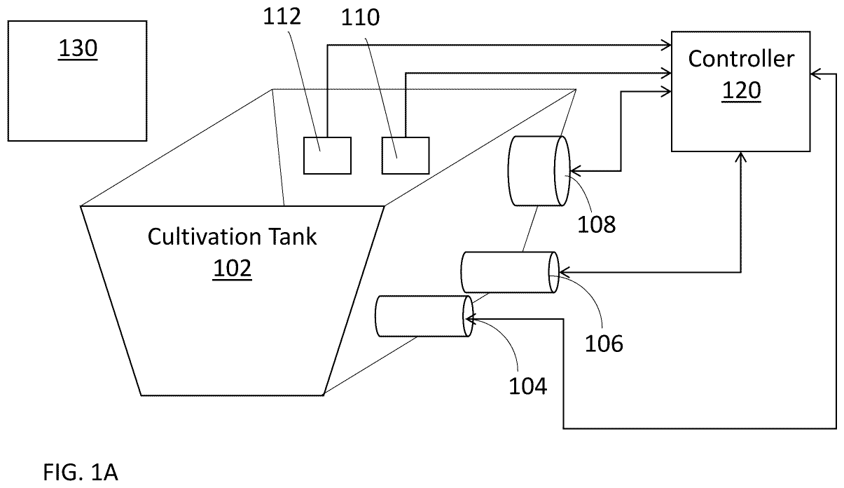

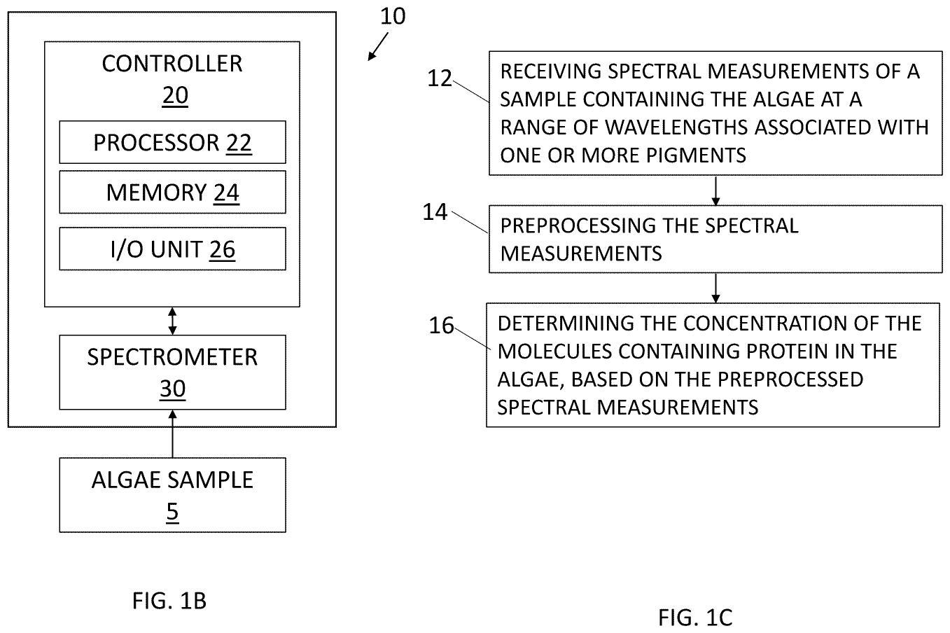

Reference is now made to FIG. 1A which is a schematic illustration of algae cultivation system 100 according to some embodiments of the present invention. System 100 may be a land-based or a sea-based algae cultivation system. System 100, may include one or more cultivation tanks 102, an air supply system 104, adapted for continuous aeration of water in the at least one cultivation tank, a water supply system 106, (e.g, seawater or artificial seawater supply system), configured to continuously supply water to the at least one cultivation tank, and a nutrients supply system 108 configured to supply nutrients, such as Ammonium (NH₄) and Phosphate (PO₄), to cultivation tank 102.

In some embodiments, one or more cultivation tanks 102 may be open conditioners (as illustrated) or closed cultivation reactors. As should be understood by one skilled in the art the invention is not limited to any specific cultivation tank or specific cultivation system.

According to some embodiments, system 100 may further include at least one light intensity sensor 110 and at least one temperature sensor 112. It should be appreciated by those skilled in the art, that additional or alternative sensors may be used in order to monitor cultivation conditions in cultivation tank 102.

System 100 may further include a controller 120, configured to control cultivation parameters based at least in part on indications received from light intensity sensor 110 and/or temperature sensor 112 (or any other sensor in system 100). System 100 may further include a temperature control unit, that may be, according to some embodiments included in water supply system 106, is or may be a separate unit including a heat source and/or a heat exchanger (not shown) to allow adjusting the temperature in cultivation tank 102 based, for example, on the temperature measured by sensor 112.

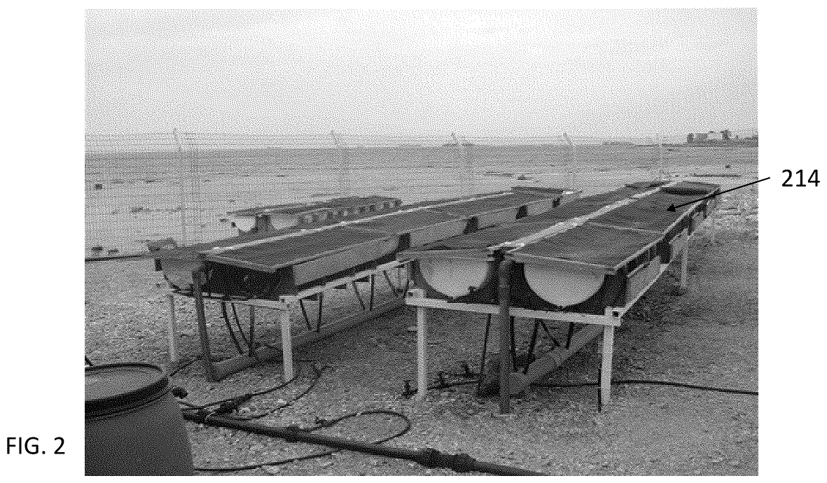

According to some embodiments, system 100 may further include a light intensity control unit, such as a net (214 in FIG. 2), e.g., 5 mm meshed plastic net. It should be appreciated that other materials and other dimensions may be used.

System 100, according to some embodiments, includes a spectral measurement unit, such as a Fourier-Transform Infrared (FTIR) spectroscopy system 130.

A study has been designed to assess the viability of marine red macroalgae of the genus Gracilaria spp. as an alternative source of edible protein in a land-based cultivation system such as the system illustrated in FIG. 1A.

Reference is now made to FIG. 1B which is a block diagram of a system for assessing the concentration of molecules containing protein in algae according to some embodiments of the invention. A computer-based system 10 may include at least a controller 20 and a spectrometer 30. In some embodiments, controller 20 may include a processor 22 that may be, for example, a central processing unit (CPU) processor, a chip, or any suitable computing or computational device. Controller 20 may further include a memory 24 may be or may include, for example, a Random Access Memory (RAM), a read-only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 24 may be or may include a plurality of possibly different memory units. Memory 24 may be a computer or processor non-transitory readable medium, or a computer non-transitory storage medium, e.g., a RAM. In one embodiment, a non-transitory storage medium such as memory 24, a hard disk drive, another storage device, etc. may store instructions or code which when executed processor 22 may cause the processor to carry out methods as described herein, for example, methods for assessing concentration total cellular protein in algae according to some embodiments of the invention.

In some embodiments, controller 20 may further include one or more input/output units 26. The input unit may be or may include any suitable input devices, components, or systems, e.g., a detachable keyboard or keypad, a mouse, and the like. The output unit may include one or more (possibly detachable) displays or monitors, speakers, and/or any other suitable output devices.

In some embodiments, system 10 may include a spectrometer 30 configured to take spectral measurements from algae sample 5. Spectrometer 30 may be a portable spectrometer capable of taking spectral measurements directly from algae cultivation system 100 or from samples taken from algae cultivation system 100. The spectral measurements from algae sample 5 may be taken on the field and analyzed in real-time by system 100 in order to assess the concentration of the total cellular protein in algae. Spectrometer 30 can detect light at a wide wavelength range across the visible IR (VIS), near-IR (NIR), and shortwave IR (SWIR), for example, from 400 nm to 2500 nm.

Controller 20 may be direct communication either by wire or wirelessly (e.g., over the internet) with spectrometer 30 and controller 120 and/or a user device (not illustrated). The assessment may be used to determine the operational parameters in cultivation system 100. For example, controller 120 may receive from controller 20 the assets concentration of the total cellular protein in algae and determine based on the assessment operational parameters of at least one of: a temperature control unit, light control unit (in closed cultivation reactors, water supply system, CO₂ and/or air supply system and the like. Alternately, controller 20 may determine the operational parameters and may send them to controller 120.

Reference is now made to FIG. 1C which is a flowchart of a method of assessing the concentration of molecules containing protein in algae. The method of FIG. 1C may be performed by controller 20, controller 120, or by any other suitable controller. In step 12 spectral measurements of a sample containing the algae at a range of wavelengths associated with one or more pigments may be received, for example, from a spectrometer such as spectrometer 30 or 130. The samples may include the alga in tank 102 of cultivation system 100 or samples taken from the tank.

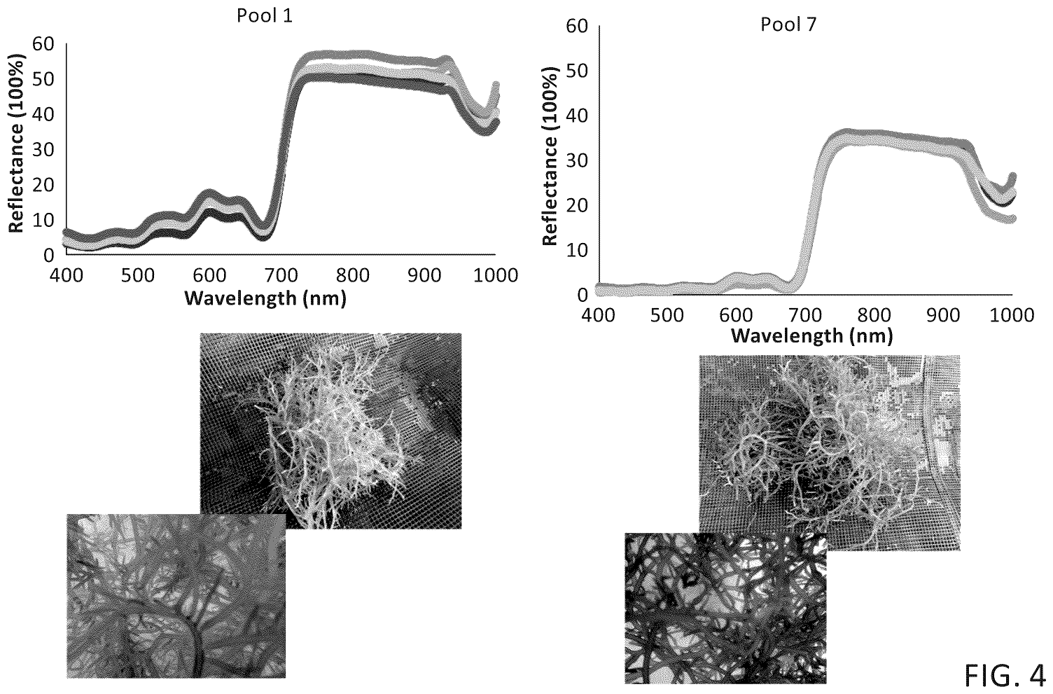

In some embodiments, wherein receiving the spectral measurements is at a wavelength range of 400 to 2500 nm, for example, between 400 to 1000 nm, between 500 to 800 nm, 450 to 810 nm, 560 to 680 nm, and any range in-between. Some non-limiting examples for such spectral measurements are given and discussed with respect to FIG. 4.

In some embodiments, the range of wavelengths associated with one or more pigments may be determined based on reflection related to molecules containing protein-related pigment that may be identified in the spectral measurements. The molecules containing protein-related pigment may be phycobiliproteins which are the main light harvesting proteinic pigments in algae, such as, Rhodophyta. Some nonlimiting examples for such phycobiliproteins may include chromophores, such as, pink/purple-colored phycoerythrin (PE), blue-colored phycocyanin (PC), and bluish-green colored allophycocyanin (APC). Examples for light harvesting complexes pigments which are contained within multiprotein complexes are including also light-harvesting chlorophyll/carotenoid-binding complexes (LHCs) a and b and xanthophylls, mainly lutein, and are referred to as Cab (for chl a/b) or LHC proteins. Reflectance graphs showing reflectance from various chromophores at 400 nm to 1000 nm are given in FIG. 4 together with images of the alga from which the spectral measurements were taken.

The pigment concentration in the sample may be calculated based on spectral measurements. For example, the diffuse reflection scattering coefficient may be calculated using the Kubelka-Munk model, using equation (1) which is an analog to absorbance transformation in transmission:

$$K / S = {( 100 - R )}^{2} / 2 * R \qquad ( 1 )$$

Wherein R is the reflectance of pigment in the algae which is related to the absorption coefficient of seaweed chromophore K and the scattering coefficient S. The ratio K/S of the coefficients is proportional to the pigment concentration in the algae. In some embodiments, the spectral measurements may be preprocessed. For example, Artificial Neural Network (ANN) may be used for the identification of minimum values, and baseline correction to smooth and normalize the scatter spectra measurements. In another example, spectral measurement repetitions of each specimen may be averaged for all measurements in order to overcome diversities due to variations in the chromophore phenotype in the samples derived from seaweed subjected to different environmental stressors (nutrient supplementation and subtraction of incident light).

In step 14, the spectral measurements may be preprocessed. For example, the spectral measurements may be filtered from background (baseline) noise using any known method.

In some embodiments, the spectral measurements may be normalized and processed using the Kubelka-Munk equation (equation 2).

$$X = ( {\sum }_{j = 1}^{n} {‖ {W}^{j} ‖}^{2} ) λ / 2 m . \qquad ( 2 )$$

where n—the number of layers, W—the weight matrix for the j layer; m—the number of inputs, and λ—the regularization parameter.

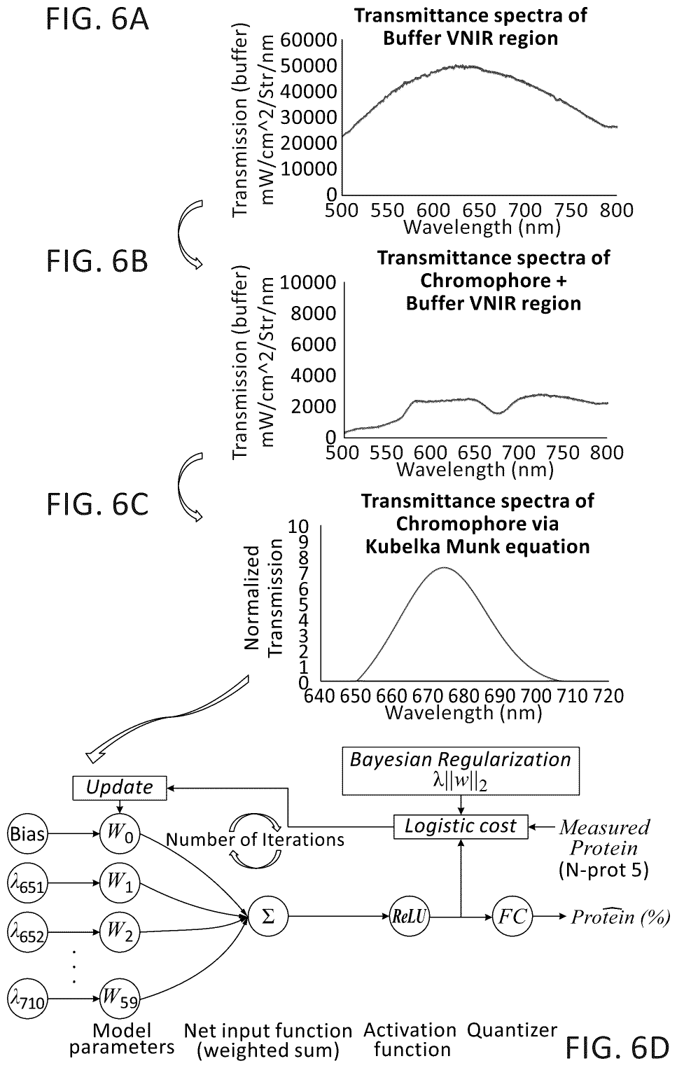

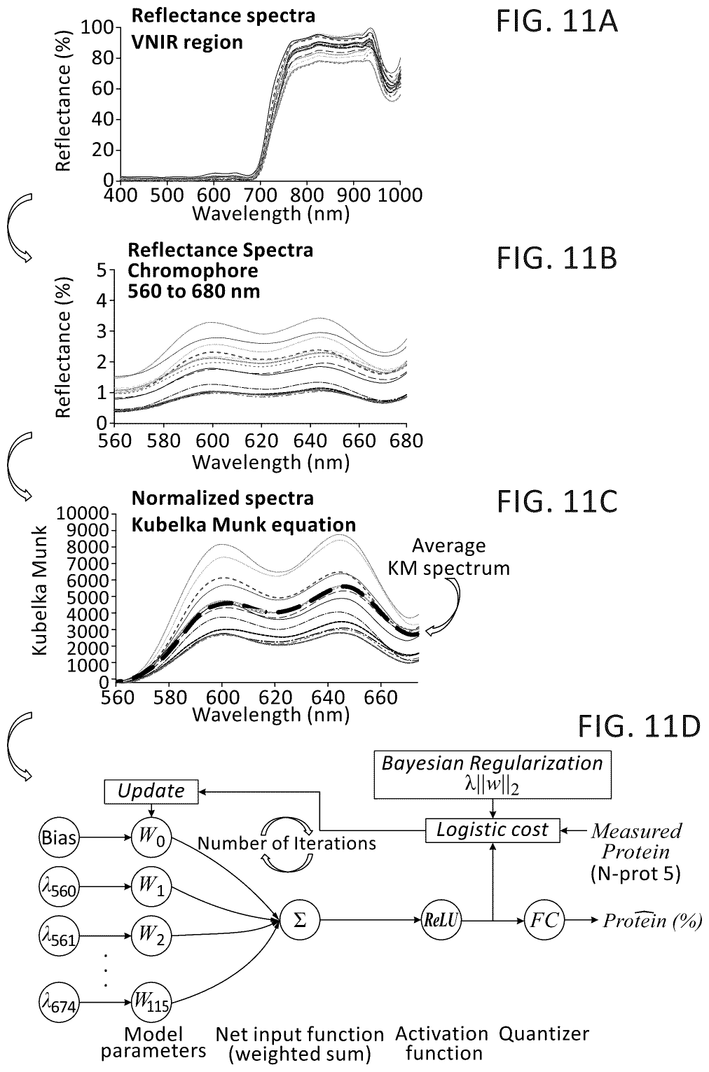

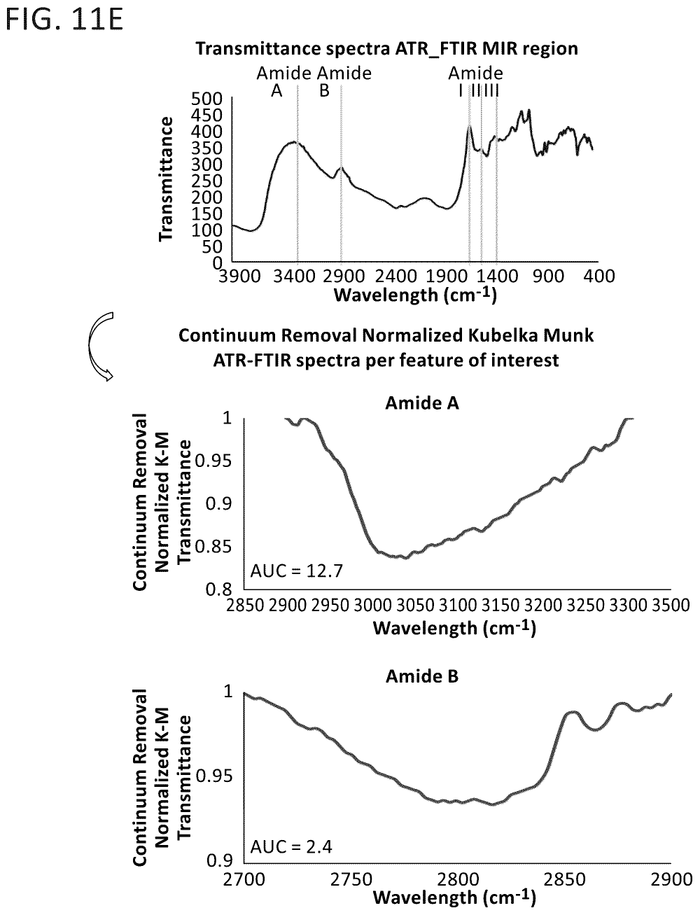

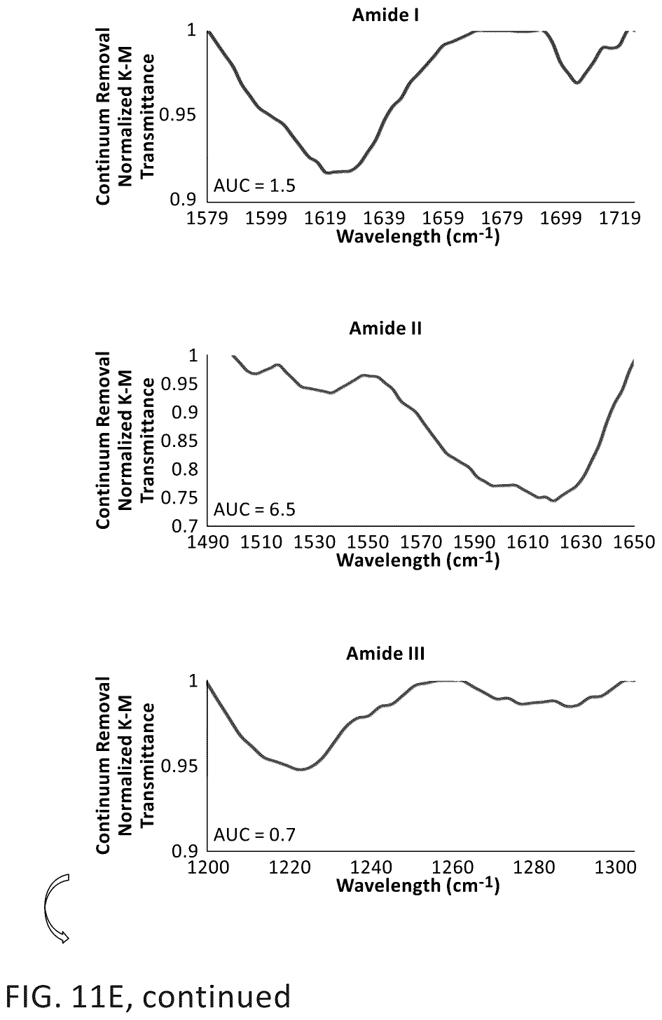

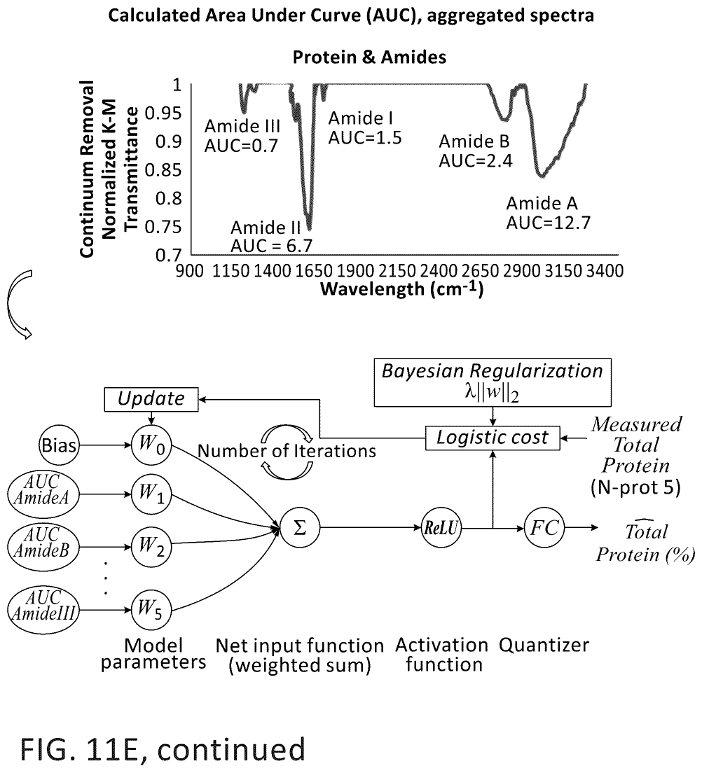

Some examples for such filtering, analysis, and calculations are shown in the graph of FIGS. 6A, 6B, and 6C which shows the raw spectral measurements taken at a range of wavelengths associated with one or more pigments of 651 to 710 nm. In FIG. 6A, the processed for specific pigment (chromophore) is shown, in FIG. 6B the filtered measurements and at FIG. 6C the preprocessed spectral measurements following the Kubelka-Munk normalization. An additional example is given in FIGS. 11A, 11B, and 11C show conducting the process to a plurality of spectra at a range of 560 nm to 674 nm which are wavelengths associated with one or more pigments.

As should be understood by one skilled in the art the filtering and normalizing methods disclosed herein are given as examples only, and the invention is not limited to filtering baseline and normalizing using the Kubelka-Munk normalization. Any method suitable for preprocessing spectral images known in the art is within the scope of this invention.

In step 16, the concentration of the molecules containing protein in the algae may be determined based on the preprocessed spectral measurements. In some embodiments, the method may include a training stage at which data associating the spectral measurements may be associated with a directly measured amount/concertation of the molecules containing protein.

In some embodiments, the direct measurements of the concertation of the molecules containing protein may be conducted by chemically analyzing the concentration/amount of nitrogen in the algae, as an indication to the amount/concentration of the molecules containing protein. The measured amount of nitrogen may be multiplied by a constant (e.g., 5) in order to determine the amount (e.g., wt. %) of the molecules containing protein in the algae (e.g., in a dry sample). From this calculation, the concertation of the molecules containing protein in the sample can be obtained.

From each algae sample used for chemically measuring the concertation of cellular protein, corresponding spectral measurements may be taken. The spectral measurements may be pre-processed as discussed above.

The chemically measured concertation of the molecule containing protein can be used to label the corresponding spectral data in order to train an ML model. In some embodiments, determining the concentration of the molecule containing protein may include applying a pretrained, first ML model on the preprocessed spectral measurements to predict the concentration of the molecules containing protein in the algae. In some embodiments, the first ML model is pre-trained based on an annotated training dataset, comprising a plurality of preprocessed spectral measurements and a corresponding plurality of annotations, representing chemical concentrations of the molecules containing protein in the algae, acquired, for example, by analyzing the concentration/amount of nitrogen in the algae, as discussed above.
annotations, representing chemical concentrations of the molecules containing protein in the algae, acquired, for example, by analyzing the concentration/amount of nitrogen in the algae, as discussed above.

In a non-limiting example, the first ML module may use a backpropagation artificial neural network (BP-ANN) to process the spectral data. BP-ANN is a multilayered network based on the generalized Woodward—BP-ANN
learning rule and weight-trained differentiable nonlinear functions with strong learning ability. The BP-ANN method was improved by introducing the momentum factor and the weight control algorithm (MBP-ANN). The MBP-ANN was conducted on the MATLAB platform. BP-ANN model-building and precision validation were implemented using the Neural Network Toolbox. The structure of the MBP-ANN was composed of three layers: input, hidden, and output layers. The pre-processed spectral data (as described above) was used as the input to MBP-ANN for the development of a cellular protein content prediction algorithm. A graphical representation of the training process is shown in FIGS. 6(d) and 11A (d).

In some embodiments, an input layer with pigment intensity per wavelength was propagated from input to output through neurons, random weights and bias, and trained, validated, and tested. In a non-limiting example, the input of one layer consisting of 10 neurons of the red wavelength absorbance spectral area (670-680 nm) was broadened to 114 neurons in 1-nm intervals to include the absorbance area of the chromophores (560-674 nm). Training methods included backpropagation to fine-tune the weights and bias of the model parameters and the Bayesian regularization technique. Since the weight and bias of the model, which depends on the hyperparameter values, can affect the model performance, the model performance was analyzed by changing the model structure and hyperparameters to ensure optimal outcomes. Therefore, the performance of the initial MBP-ANN model was optimized by changing its hyperparameters, and structural Bayesian optimization was employed to determine the optimal combination of hyperparameters to enhance the prediction performance. Based on the Bayes Theorem, a surrogate model, which is a probabilistic model of the objective function, searches for specific hyperparameters yielding the maximum or minimum performance. A rectified linear unit (ReLU) propagated backward computed errors to update the model parameters, and then the predicted normalized means were compared to the actual protein content results obtained from the CHNS elemental analytical analysis.

MBP-ANN training, testing, and validation of the algae thallus phenotype may be conducted on data obtained from the field measurements normalized and processed using the Kubelka-Munk equation (Eq. 2) with 1000 iterations until fully connected. In a non-limiting example, the input data set for the MBP-ANN consisted of 164,160 spectral measurements of which 70% were used for training, 15% for testing, and 15% for validation. The output data was the prediction of protein content (% DW) that fully connected with pigment intensity per wavelength within the VIS-NIR area (560-674 nm).

In some embodiments, a similar methodology can be applied for at least one type of: molecule containing protein in the algae. Therefore, the method of FIG. 1C may further include identifying in the preprocessed spectral measurements at least one type of molecules containing protein. In some embodiments, spectral measurements may be taken from a plurality of algae samples. For each sample, an FTIR analysis (e.g., Attenuated Total Reflection (ATR) FTIR analysis) may be conducted for identifying the type of molecule containing protein in the algae. The identified type of the molecules containing protein may be used as a label in training the ML model to also predict the type of the protein and/or the proteinaceous fraction of the cell. In some embodiments, identifying the types of molecules containing protein may include performing FTIR spectroscopy analysis on spectral measurements to produce identification of types of molecules containing protein and applying a pretrained, second ML model on the preprocessed spectral measurements, to predict at least one of the type of molecules containing protein in the alga. In a non-limiting example, the second ML module may use a backpropagation artificial neural network (BP-ANN) to process the spectral data, and may be substantially similar to the first, with the required changes of the training data.

### Experimental Results

The following are non-limiting examples for assessing protein concentration in algae. A similar methodology can be applied for assessing the protein fraction concentration in algae. The goal of the experimental setup was to assess the viability of marine red macroalgae of the genus Gracilaria spp. as an alternative source of edible protein. This Rhodophyta species, known for its rapid growth rate and commercial value, accounted of 10.7% of the world seaweed produced in 2018. Gracilaria spp., is also the most important source for agar production and a source of sulfated polysaccharides which are used in pharmaceutical and biotechnology industries. To meet this goal, a real-time spectrometric tools intended to aid farmers in protein yields management and harvest time accuracy in land-based seaweed farming, is required. Such spectrometric tools should be designed to quantify seaweed protein (i.e., functional and structural protein) related to photosynthetic pigments.

### Methodology

### Growing the Algae

Land-based seaweed cultivation protocol, improved protein aggregation and harvest time optimization
Hypothesis:

The hypothesis may include that large amounts of nutrients, in particular nitrogen, will favor the biomass yield and protein content in the investigated seaweeds. Large nutrient amounts are typically found in polluted marine environments or close to aquaculture (fish) settings. Hence, nutrient consumption by seaweeds can have additional environmental benefits in addition to protein production. We also hypothesize that protein aggregation relates to light intensity and seaweed thalli pigmentation due to protection mechanism of the seaweed against the harmful effects of excessive incident light. Therefore, protein content and seaweed thalli pigmentation variability are expected.
Experimental work:

An experimental, land-based seaweed cultivation system, as seen in FIG. 2, was used for biomass production and productivity assessments. The culture system may include, for example, 18, 40 L tanks each with continuous aeration and seawater supply. Gracilaria spp. conferta, known to contain substantial amounts of proteins in nature and found in the intertidal zone of the Israeli Mediterranean Sea has been used as a model species. The seaweeds were supplied with nutrients (NH₄ and PO₄) and the tanks covered with plastic nets (meshed 5 mm) to control incident light. Data-loggers (HOBO) for temperature and light intensity were installed in the tanks.
Experimental setup:

Seaweeds have been cultivated with stocking densities of 1 kg FW m⁻². About 100 g fresh weight (FW) of Gracilaria spp. was stocked at start at each tank. The algae was grown for 3-4 consecutive weeks under a nutrient supply regime (see below) during a year to also capture seasonal effects. Growth rates, which can be translated into biomass productions rates, have been determined. Growth rates (Specific Growth Rates, SGR) may be established by measuring the FW of the seaweeds every week or two, and calculated using the following formula (equation 3),

$$SGR ( \% ) = ( ln ( \frac{{w}_{t}}{{w}_{0}} ) \times 1 \; 00 ) / t \qquad ( 3 )$$

where W₀ is the initial biomass and Wₜ is the biomass after t culture days and will expressed as % FW per day.

The experimental setup consisted of six treatments (including control) each with three replications, totaling 18 tanks. One treatment received regular seawater lacking addition of external nutrients (T-1); a second treatment received N and P at concentrations of 1 and 0.1 mM, respectively (T-2). Nutrients have been added twice a week for 24 h to allow their capture by the seaweed. The third set of three tanks (T-3) received N and P at concentrations of 2.0 and 0.2 mM, respectively, imitating the nutrient loads from fish cultivation. Treatments were covered with single layer (n=9) or double layer (n=9) 5 mm mesh net. Biomass samples have been collected every other week for protein aggregation quantification expressed as % of dry weight.
Expected results:

Seaweed biomass was expected to respond with high yields and high protein content when treated with high nutrient supply. Experiments seek to develop a precise cultivation protocol, smoothing up protein aggregation and to harmonize protein quantity and quality and production sustainability and profitability.

### Collecting Spectral and Analytical Data

Precision color indices algorithm (PCIA) for a rapid protein quantification in seaweed biomass.
Hypothesis:

Photosynthetic pigments capture sunlight to supply energetic needs in photosynthesis. Significant components of these pigments are structural proteins and sugars. In red seaweeds, phycobiliproteins (a family of light-harvesting pigment protein complexes) may represent 20% of dry matter and 50% of water-soluble proteins. Therefore, we hypothesize that seaweed thalli visible color is correlated with protein prevalence in the biomass and can be a reliable indicator for protein concentration.
Experimental work:

For an early spectral investigation aiming to assess Gracilaria spp. thalli pigmentation, a set of on-site and in-lab spectral and imagery measurements have been ran and for protein quantification in-lab analytical and spectroscopy measurements has been ran.

A field spectrometer using a portable acquiring data across the VIS and NIR range from 350 to 1100 nm with a resolution of 0.5 nm and an accuracy of 1 nm was used for on-site reflectance, and later for in-lab transmittance measurements. Spectrometer calibration was done against a white Spectralon plate. Bare fiber optic with 25° field-of-view positions were used for detailed spectra collection. A portable digital camera was used for imagery technique. FT-IR system in Attenuated Total Reflectance (ATR) mode was used as a quantitative protein molecule tool. The images were taken at a resolution of 8 cm⁻¹ in a spectral range of 4000-748 cm⁻¹ and 16 or 32 scans per spectrum.

CHNS element analyzer was used for total nitrogen analysis. Calibration was done against organic nitrogen and carbon standards, such as caffeine, L-glutamic acid and sulfamic acid. The nitrogen content of the dried samples was measured using a Flash 2000 Organic elemental analyzer (Thermo Scientific, USA). About 2-3 mg of the dried and milled material from each specimen (n=144) were combusted at 960° C. according to manufacturer's protocol. The nitrogen content of the dried samples was used to determine protein content, assuming a nitrogen-to-protein (N-prot) conversion factor of 5.0, which has been shown to be appropriate for marine seaweeds.
Experimental set-up:

At first, on-site spectral reflectance was measured directly on seaweed thalli on a weekly basis using seaweed samples (FW) from each tank (n=18). The reflectance measurements were analyzed in the visible (VIS) spectrum within the wavelengths range 580-710 nm of the red band—which classified the red (Rhodophyceae) seaweed group and Gracilaria spp. as well. The detailed spectra were collected at a nadir view angle approximately 5-10 cm above the seaweed thalli scale by a bare fiber optic with 25° field-of-view positions. Measurements were repeated several times. For visible color indicis imagery technique were used. Seaweed samples from each tank (n=18) were photographed on a weekly basis submerged in 40 L pool and on top the net layer using the portable digital camera. For on-lab transmittance measurements pigment from all samples (n=18) were extracted on a weekly basis. The extraction procedure included approximately 1 g of fresh biomass manually milled with the use of mortar and pestle. The fresh and crushed samples from each treatment were dissolved in 6 ml deionized water media, and were filtered through 0.45 μM filter and the supernatant was transferred to a lock tube. The transmittance spectra (log 1/T) in the visible NIR region was obtained using the portable filed spectrometer scanning the samples between 450 and 2500 nm at a speed of nm min⁻¹ and an interval of 2 nm. Regular transmittance was read at 580-710 nm. Fourier transform infrared (FTIR) spectroscopy measurements were used as a quantitative protein molecule tool. Samples of fresh seaweed thalli from each treatment (n=18) were collected on a weekly basis and were dried in a room temperature and milled manually with the use of mortar and pestle. The dried samples were analyzed using a FT-IR system in Attenuated Total Reflectance (ATR) mode. The images were taken with a spectral resolution of 8 cm⁻¹ in a spectral range of 4000-748 cm⁻¹ and 16 or 32 scans per spectrum.

In general IR spectra of proteins characteristics have absorbance bands peak at wavenumber 1600-1700 cm⁻¹ (Amide I); 1500-1600 cm⁻¹ (Amide II); 1200-1300 (1400) cm⁻¹ (Amide III) and ˜3300 and ˜3070 cm⁻¹ (Amide A and Amide B). The IR region (NIR/SWIR), the shape of the signal (broad/sharp) and the intensity (weak/medium/strong) are using as a fingerprint for protein prevalence identification. Here bands of interest according to literature, are 1000-1096 cm⁻¹; 1099-1176 cm⁻¹ and 1179-1685 cm⁻¹. To determine the protein content, total nitrogen in the samples from each treatment (n=18) was quantified using CHNS element analyzer. Common organic nitrogen and carbon standards, such as Caffeine, L-glutamic acid and Sulfamic acid were used for calibration. Fresh seaweed from each treatment was stored in sealed plastic bag at −70° C. Prior to analyses, thalli were rinsed with tap water, dried overnight in an oven at 60° C. and were grounded to a granulated particle using a coffee grinder machine. A few mg of the dried and granulated particles from each treatment (n=18) were combusted at 925-1,000° C. The nitrogen percentage of the dry weight was used to calculate the protein content in the dried biomass according to a nitrogen-to-protein conversion factor of 5.
Expected Results:

It was expected that all measurements data (imagery, spectral and analytical) will be varied in accordance to seaweed visible color thalli and protein prevalence in the biomass. For processing raw infrared spectra data, a MATLAB was used and for modeling and analysis we will review different statistical methods to model precision color indices algorithm (PCIA) as a prediction tool for protein aggregation in the seaweed biomass.

### Assessing the Amount of Protein

Outlining machine learning (ML) techniques for an accurate protein content prediction and yield estimation at early stage of seaweed cultivation.
Hypothesis:

Quantitative prediction of protein yield at early cultivation stage is a valuable mean to support decision management and improve desirable outcome. At this stage we will outline a ML algorithm capable of analyzing large datasets of information to learn about the aggregated protein in the seaweed biomass, combining information on cultivation strategies and processes with environmental, economic, and expert knowledge (Chlingaryan et al. 2018). We hypothesize that the ML tool will be able to assess at early stage which of the features is more informative for protein yield, will improve protein yield productivity and quality from seaweed, and will reduce operational and external costs (Finger et al. 2019).
Experimental work:

Different ML approaches recommended in the literature for site-specific management have been tested to achieve the most accurate protein yield prediction. Artificial Neural Networks, Support Vector Machine, Partial Least Squares Regression, and Fuzzy Cognitive Maps that also can be used to model expert knowledge for yield prediction and crop management are some examples.

Data collection focus on: data from the experimental work (phase 1) and from imagery, spectral and analytical measurements (phase 2); data related to environmental influences and economic aspects including GHG emissions during production; data derived from expert knowledge analysis.
Experimental setup:

Based on collected data we developed variable selection mechanisms and measurements. In addition, we applied a statistical method for variable weight to understand which of the variable parameters strongly affect protein yield for resource use efficiency and risk reduction. At first, we have taken as input the desired cultivation parameter to maximize protein aggregation: cultivation time before harvest, biomass density, daily growth rate, incident light, temperature, nutrients supplement regime, methods to smooth the effect of seasonal and environmental fluctuations. Visible seaweed desirable thallus pigment was selected according to imagery data analysis and desirable spectral VIS/NIR reflectance and transmittance wavelength after calibration. Pigment measurements were analyzed with respect to ATR-FTIR spectroscopy measurements and analytical analysis to assess seaweed specific changes of protein content. The environmental impact analysis considered the assessment of greenhouse gases emissions and cost estimates for practicing on-land seaweed production. Expert knowledge was considered to address different aspects that could affect the willingness to adopt seaweed as a preferable protein source for food. We conducted an in-depth interview and assess the results by multicriteria evaluation tools. We developed variable selection mechanism and measurements and applied statistical methods for variable weight to understand which of the variable parameter strongly affect protein yield quality and productivity, resource use efficiency and risk reduction.
Expected results

Simplify application for the seaweed grower to better manage protein yield on a seaweed biomass-based or pool-based level.

### Results

The Land-based seaweed cultivation protocol, improved protein aggregation and harvest time optimization.

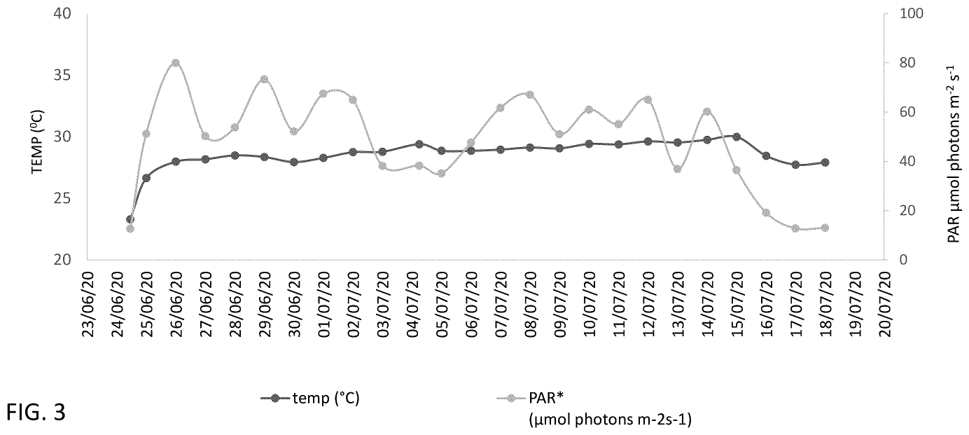

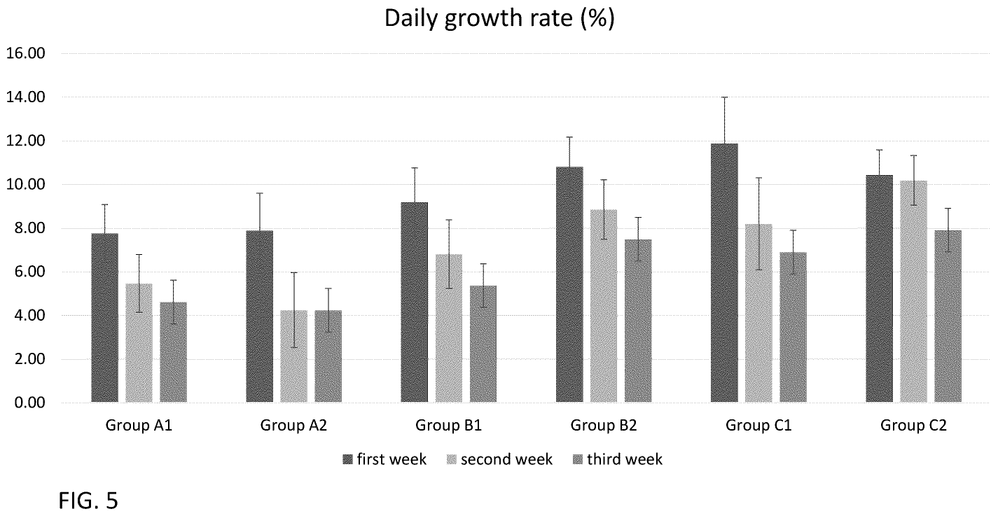

As may be seen in FIG. 3, during the three consecutive weeks of summer cultivation in 2020, the average seawater temperature was 28° C. and average daily irradiance 48 μmol photons m⁻² s⁻¹. The biomass was weighted every week and the yield was harvested. Biomass in six treatments showed the same daily growth rate pattern with the highest growth rate during the first week and then declined continuously (as seen in FIG. 5).

Average growth rate was insignificantly different between treatments (n=6) according to One-way ANOVA (α=0.05) test, but was significantly different between pools (n=18).

An explanation for the incompatibility between ANOVA test results might be confounding factors such as differences in the physical position and color variability of the pools that might affect light irradiance, biomass photosynthesis and growth rate within treatments. According to literature significant difference of means between similar treatments have been achieved before at the same location. Therefore, during the next seasons of the trail, a net cover common to all treatments will be apply for light irradiance control. Also, nutrient addition and harvest time will be optimized.

Seaweed thalli visible pigmentation variation was demonstrated on a pool-basis (n=18) both by portable camera imagery output (FIG. 4) and spectral measurements (e.g., red band reflectance measurements of the VIS spectrum 580-710 nm, (FIG. 6).

TABLE 1
ANOVA: Multi Factor test between treatments (1) and between pools (2)

| Source of Variation | | | | | |
|---|---|---|---|---|---|
| SS | df | MS | F | P-value | F crit |

ANOVA (1)

| | | | | | | |
|---|---|---|---|---|---|---|
| Between Groups | 45.74123721 | 5 | 9.148247 | 2.47424684 | 0.092185 | 3.105875 |
| Within Groups | 44.36864073 | 12 | 3.697387 | | | |
| Total | 90.10987794 | 17 | | | | |

ANOVA (2)

| | | | | | | |
|---|---|---|---|---|---|---|
| Between Groups | 190.3667226 | 17 | 11.19804 | 2.482908 | 0.010747 | 1.915321 |
| Within Groups | 162.3618413 | 36 | 4.510051 | | | |
| Total | 352.7285639 | 53 | | | | |

Multivariant linear regression was used to demonstrate possible relation between Fourier transform infrared (FTIR) spectroscopy measurements and protein content in the seaweed biomass derived from the CHNS elemental analysis.

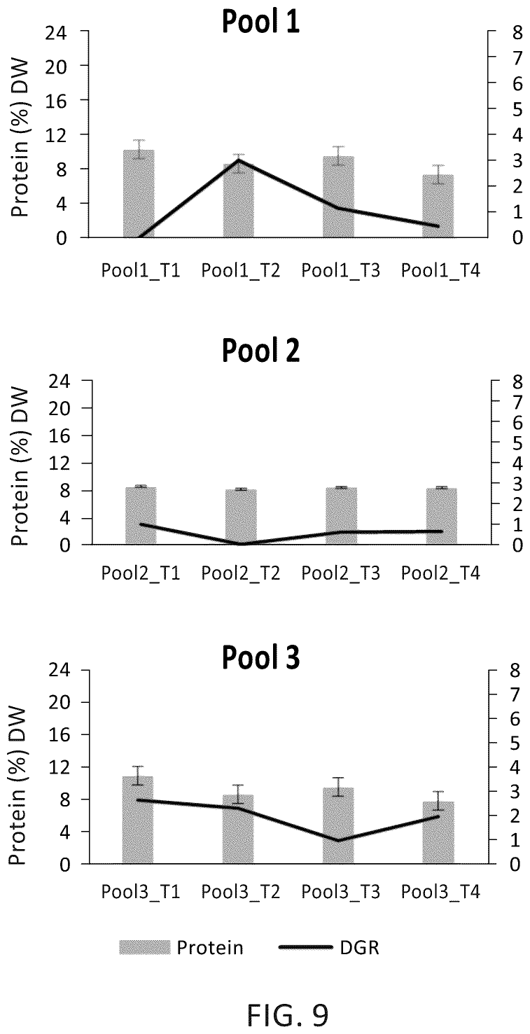

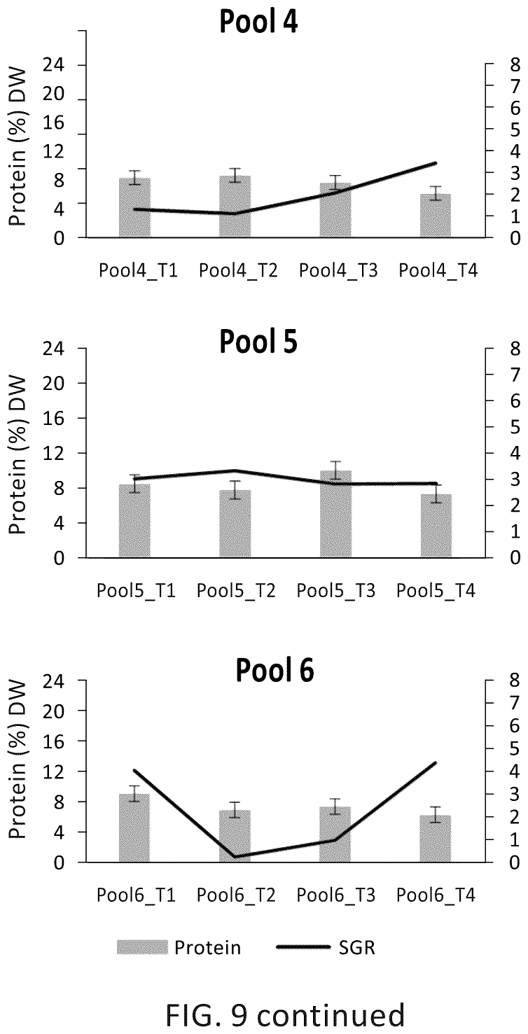

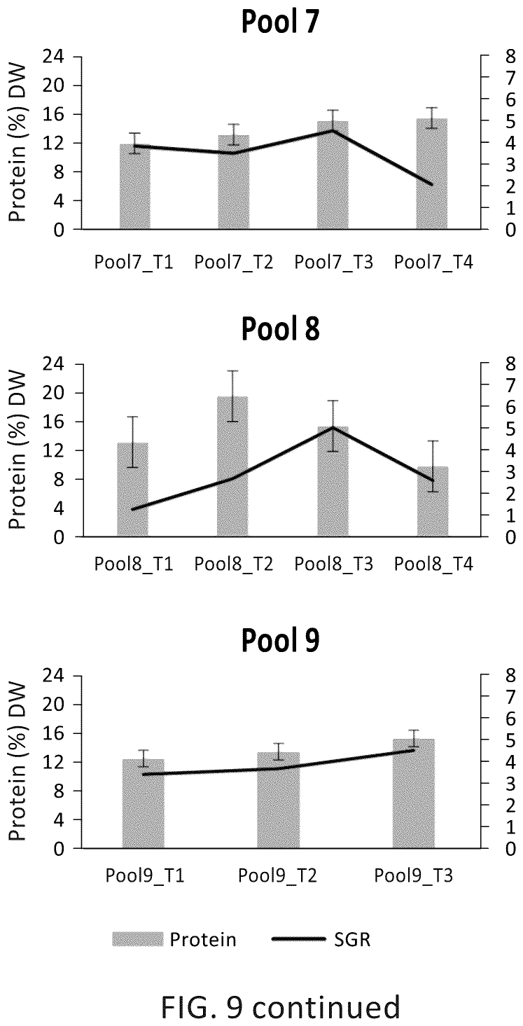

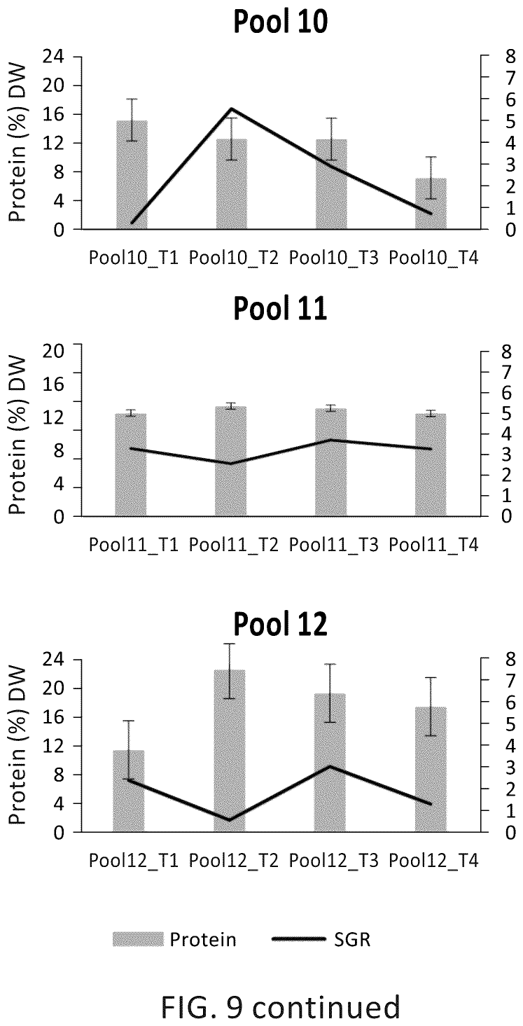

Protein content determination (%) of the dry matter was quantified using CHNS Flash 2000 Organic elemental analyzer multiplied by a nitrogen-to-protein conversion factor that is relevant to red seaweed. The results of the protein content determination are presented in FIG. 9.

Reference is now made to FIG. 9 which is a comparison of algae growth rate and protein content. Specific growth rate (SGR) measured as FW (%/day) over four consecutive weeks, in 18 pools, in comparison with protein content (% DW) for the cultivated Glacilaria spp during fall season. Data was analyzed per specimen per container (n=18) and figures are in average±SD (error bars).

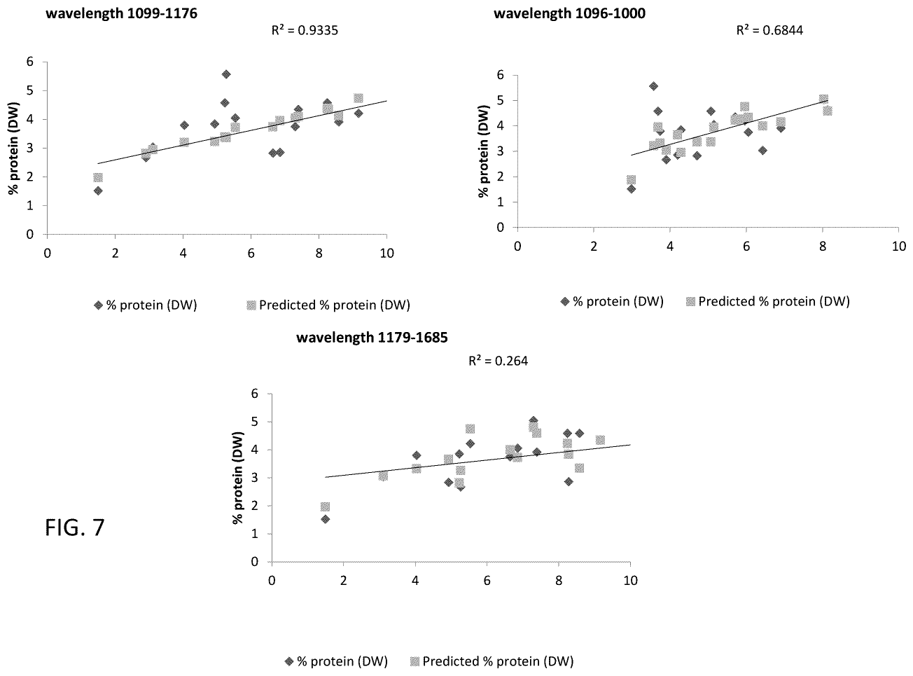

FTIR spectra intensity was determined as the normalized area under the peaks within spectral ranges: 1685-1179 cm⁻¹; 1099-1176 cm⁻¹; 1096-1000 cm⁻¹ which are diagnostic bands for protein. Predicted values of protein compounds of interest were plotted against their values derived from the classical chemical analysis. Good predictive performance (R²>0.93) recorded for band intensity within the range 1099-1176 cm⁻¹; moderate correlation (R²>0.68) within the range 1096-1000 cm⁻¹ and low prediction accuracy (R²>0.26) within the range 1179-1685 cm⁻¹ (FIG. 7).

FTIR spectrum corresponding to protein molecules is highly complex due to secondary structure of the protein molecules which impact the molecule interaction with IR light and also overlapping bands in the absorption spectrum of proteins that limit the information that can be obtained. The precision color indices algorithm (PCIA) as a prediction tool for protein concentration in the seaweed biomass that is developing for the first time, may rely upon many measurements' repetitions. Group of indices that already developed to estimate concentration of leaf pigments by correlating visible pigments and laboratory spectral measurements may be screened and the applicability of the most relevant indices may be tested for optional use with required modifications.

### Additional Results

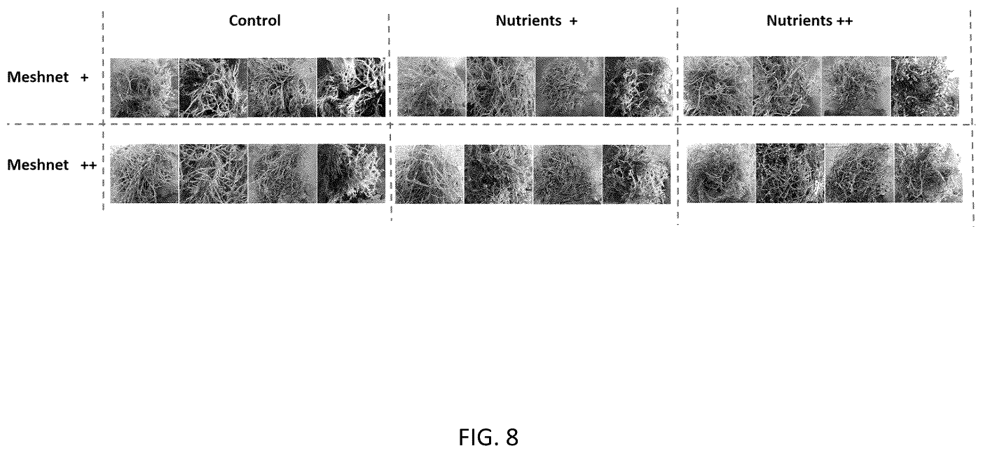

Reference is now made to FIG. 8 shows images of Cultivated Gracilaria spp. Specimens showing changes in reflectance due to changes in nutrients/fertilizing regime and incident light exposure. Therefore, the proposed prediction can also predict/assess the influence of nutrients/fertilizing regime and incident light exposure on the protein molecule concentration.

Reference is now made to FIGS. 11A, 11B and 11C which show the preprocessing of various algae samples showing the raw spectral measurements taken in the VNIR spectrometer at 400 to 1000 nm at the graph in FIG. 11A. Only the relevant portion of the spectrum from 560 to 674 was identified as a range of wavelengths associated with one or more pigments. The identified spectral data was filtered from background (baseline) noise at graph in FIG. 11B to be normalized using the Kubelka-Munk equation presented at the graph in FIG. 11C. The results were used to train the first ML model. Training process is illustrated in FIG. 11D in which the vector consisting of intensities in various wavelength is provided to the ML model labeled with measured protein concentration measured from N content. In each iteration the ML model changes the weight provided to each wavelength until the predicted protein % fits the measured protein % (wt. %) as discussed with respect to step 16 of the method of FIG. 1C.

Reference is now made to FIG. 11E which is shown FTIR spectral measurements and normalized FTIR spectral measurements associated with five types of amides (Amide A; Amide B; Amid I; Amide II; Amide III). The use of FTIR spectroscopy allows to distinguish between the different types of Amides.

### Post-Processing and Validation

Reference is now made to FIG. 7 which shows a comparison between the chemically measured protein, measured using FTIR, and the predicated protein, measured and predicted according to embodiments of the invention discussed above. As clearly shown from the graph, there is a strong correlation between the predicted and measured protein, showing that the predicted model can be used for simple on-field measurements of protein in algae, based on spectral measurements.

In other to quantitively validate the model, an external validation trial was performed for four consecutive weeks during the fall of 2021 for six cultivation regimes (including control). To smooth specimen variability due to abiotic stress, the trial setting was narrowed to six containers, and three replicates of Gracilaria sp. were taken randomly from each container (i.e., 18 samples). To assess the ability of MBP-ANN to automatically extract useful patterns and predict protein content from a data set that was not previously used, Fifty-four samples of the cultivated fresh biomass were classified in-situ via reflectance spectral features measurements (560-680 nm) using absorption depth. The input data included field measurements on seaweed thallus phenotype, normalized and processed using the Kubelka-Munk equation. The predicted normalized means were compared to the actual protein content results obtained from N content elemental analysis of the dried samples, which were converted to protein content (% 377 DW) using the same N-prot factor of 5.0. Model performances were assessed in terms of the regression coefficient R², the mean square error (MSE) and the root mean square error (RMSE) for the validation results, according to the following equation (4)

$$RMSE = \sqrt{\frac{1}{n} {\sum }_{i = 1}^{n} {( \hat{y} i - {y}_{i} )}^{2}} \qquad ( 4 )$$

where yi, ŷi—measured and predicted values of protein content, and n—the number of samples.

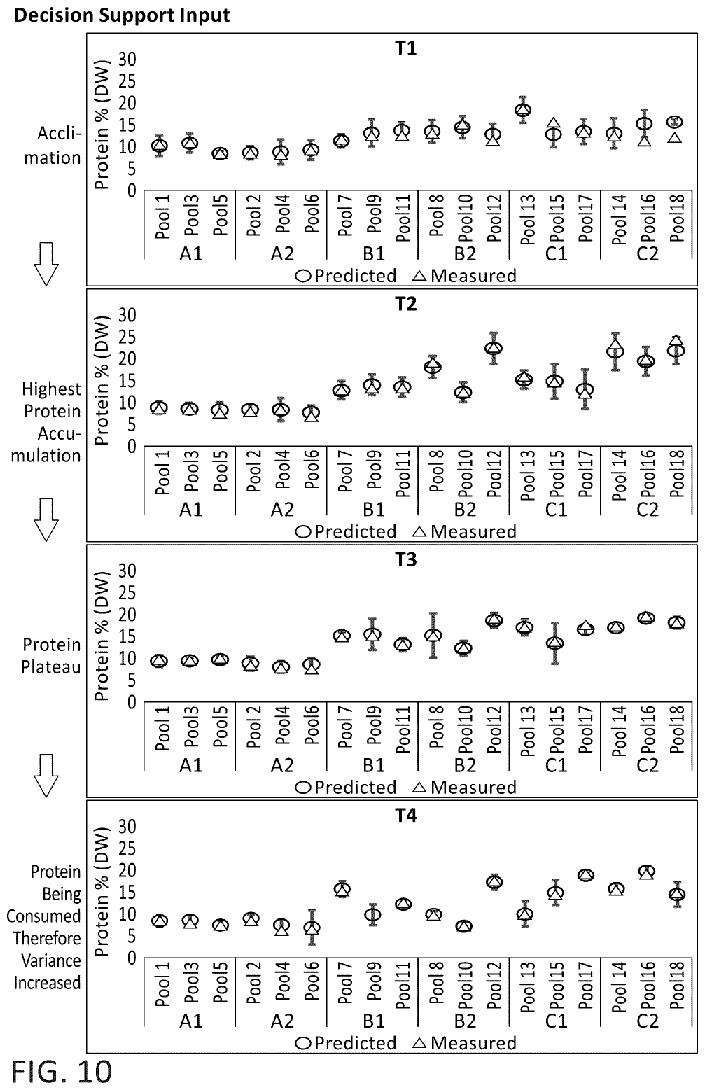

The MBP-ANN was trained with 1000 iterations to connect pigment intensity with protein absorption in a narrow zone of the red wavelength 670-680 nm resulted in relatively low prediction accuracy of R²=0.74. Broadening the absorption area to include chromophores at 560-674 nm for data training produced much higher values of the correlation coefficient (R²). The initial optimal value of mu was 0.9899, and the optimal number of hidden neurons, which are structural variables for the model, was 10, as shown in Table 2. The optimized model performances for the training and test data were compared with the performance of the initial model, as shown in table 3. The R² for the test data was 0.95 (p<0.01) for the optimized model, as shown in FIG. 10. Moreover, 423 the RMSE for the optimized model was 0.84 indicating a higher accuracy than for the initial 424 model (RMSE=4.6, table 3).

TABLE 2
Hyperparameters for MBP-ANN optimization

| Hyperparameter | Description | Optimized value |
|---|---|---|
| Neuron size | Number of neurons in | 10 |
| | the hidden layer | |
| mu | Initial mu setting | 0.9899 |
| mu decrease | mu decrease factor | 0.8692 |
| mu increase | mu increase factor | 6.8909 |
| Min gradient | Minimum performance gradient | 6.45E−06 |

TABLE 3
Performance analysis for the MBP-ANN prediction model

R² RMSE

| | | |
|---|---|---|
| Initial model Training | 0.92 | 3.85 |
| Test | 0.89 | 4.6 |
| Optimized model Training | 0.97 | 0.64 |
| Test | 0.95 | 0.84 |

Prediction accuracy for results of initial and optimized model.
R² = coefficient determination, RMSE = root mean square error.

Reference is now made to FIG. 10 which shows model prediction performances. Mean±standard error of predicted protein content values in the algae biomass (% DW) as obtained from the MBP-ANN model, in comparison with laboratory results obtained from the CHNS analytical analysis of N content converged to protein content. The graphs present measurements during four consecutive weeks (hereafter designated T1-T4) conducted during the fall season of 2020. Range of R²: 0.718-0.991; Range of MSE: 2.23-0.23; Range of RMSE: 1.49-0.48.

Unless explicitly stated, the method embodiments described herein are not constrained to a particular order in time or chronological sequence. Additionally, some of the described method elements may be skipped, or they may be repeated, during a sequence of operations of a method.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of assessing the concentration of molecules containing protein in algae comprising:
receiving spectral measurements of a sample containing the algae at a range of wavelengths associated with one or more pigments;
preprocessing the spectral measurements; and
determining the concentration of the molecules containing protein in the algae, based on the preprocessed spectral measurements,
wherein the molecules containing protein consist of at least one or more of; protein and protein bounded to one or more nonprotein molecules.

2. The method of claim 1, wherein the range of wavelength associated with one or more pigments is determined by identifying in spectral measurements a range of wavelength associated with one or more pigments.

3. The method of claim 1, wherein determining the concentration of the molecule containing protein is based on chemical measurements of the amount of molecule containing protein associated with spectral measurements, stored in a database.

4. The method of claim 3, wherein the chemical measurements are nitrogen concentration measurements in the algae.

5. The method of claim 2, wherein determining the concentration of the molecule containing protein comprises applying a pretrained, first machine-learning (ML) model on the preprocessed spectral measurements to predict the concentration of the molecules containing protein in the algae.

6. The method of claim 5, wherein the first ML model is pre-trained based on an annotated training dataset, comprising a plurality of preprocessed spectral measurements and a corresponding plurality of annotations, representing concentrations of the molecules containing protein in the algae.

7. The method of claim 1, further comprising, identifying in the preprocessed spectral measurements least one type of molecules containing protein.

8. The method of claim 7, wherein the identification is based on Attenuated Total Reflection (ATR) Fourier Transform Infrared (FTIR) spectroscopy analysis associated with preprocessed spectral measurements, stored in a database.

9. The method according to claim 7, wherein identifying comprises:
performing FTIR spectroscopy analysis on spectral measurements to produce identification of types of molecules containing protein; and
applying a pretrained, second ML model on the preprocessed spectral measurements, to predict at least one of the type of molecules containing protein in the alga.

10. The method of claim 1, wherein the pigment is phycobiliprotein, chlorophyll/carotenoid-binding complexes (LHCs) a and b, and xanthophylls.

11. The method of claim 10, wherein the phycobiliproteins is selected from: pink/purple-colored phycoerythrin (PE), blue colored phycocyanin (PC) and bluish-green colored allophycocyanin (APC).

12. The method of claim 1, wherein receiving the spectral measurements is at a wavelength range of 400 to 2500 nm.

13. The method of claim 1, wherein receiving the spectral measurements is at a wavelength range of 500-800 nm.

14. The method of claim 1, further comprising calculating the pigment concentration is based on calculating a ratio between a reflectance coefficient and the scattering coefficient from the spectral measurements.

15. The method of claim 1, further comprising:
determining growth parameters for growing the algae based on the determined protein concentration and protein-fraction concentration.

16. A system for assessing concentration of total cellular protein in algae, comprising:
a spectrometer; and
a controller configured to:
receive spectral measurements of a sample containing the algae at a range of wavelengths associated with one or more pigments;
preprocess the spectral measurements; and
determine the concentration of the molecules containing protein in the algae, based on the preprocessed spectral measurements,
wherein the molecules containing protein consist of at least one or more of; protein and protein bounded to one or more nonprotein molecules.

17. The system of claim 16, wherein the spectrometer is a portable wide range spectrometer.

18. The system of claim 17, wherein the portable wide range spectrometer measures spectrum at 400 to 2500 nm.

19. The system of claim 16, wherein the range of wavelength associated with one or more pigments is determined by identifying in spectral measurements a range of wavelength associated with one or more pigments.

20. The system of claim 16, wherein determining the concentration of the molecule containing protein is based on chemical measurements of the amount of molecule containing protein associated with spectral measurements, stored in a database.

21.-31. (canceled)
